(19) United States
(12) Patent Application Publication
(10) Pub. No.: US 2013/0034892 A1
(43) Pub. Date: Feb. 7, 2013

(54) METHOD FOR THE PRODUCTION OF A FERMENTED NATURAL PRODUCT

(71) Applicant: Cordula Niedermaier-may, Hohenbrunn (DE)

(72) Inventor: Cordula Niedermaier-may, Hohenbrunn (DE)

(21) Appl. No.: 13/641,028

(22) PCT Filed: Apr. 21, 2010
(86) PCT No.: PCT/EP10/55267

§ 371 Date: Oct. 12, 2012

(51) Int. Cl. C12P 1/04; C12M 1/00

(52) U.S. Cl. 435/170; 435/289.1

(57) ABSTRACT

What is described is a process for producing a fermented natural product, wherein several ferment extracts are produced from raw materials of the natural product and admixed with a propagate from microorganisms. Also described is a plant for performing the process.

### FIELD OF THE INVENTION

The present invention relates to a method for the production of a fermented natural product, in particular the production of a highly effective fermented natural product having a long shelf life. The invention further relates to a system for carrying out said method.

### BACKGROUND OF THE INVENTION

Products representing products from nature are denoted as natural products, such as milk from animals. However, natural products are also those products produced from naturally occurring raw materials, such as fruit and vegetables.

In recent years there has been an increased emphasis on a particularly healthy, balanced diet. In this regard the consumption of natural products plays an ever increasing role.

It is known to produce such natural products by means of fermenting a naturally occurring raw material, or a mixture of various raw materials in the presence of microorganisms. Such a ferment extract is then purified, such as by means of centrifugation and/or filtration, in order to remove any insoluble components. When indicated, the ferment extract is pasteurized in order to deactivate the microorganisms.

Such method is known from EP 1 153 549 B1. In this method a mixture of fruit, vegetables and legumes as raw materials is fermented in the presence of microorganisms for the production of a ferment extract, subsequent to which part of the ferment extract is subjected to at least one further fermentation process, and said part is mixed with the remaining part of the first ferment extract. After mixing, the product is heated to about 80° C., and filled into suitable containers, which are then hermetically sealed in a commonly known manner.

It has been found that the natural product produced according to EP 1 153 549 B1 has a high antioxidative potential in the form of polyphenols, thus representing protection from free oxygen radicals in the body. Furthermore, it has been determined that the product strengthens the immune defense in the body by means of enzyme activation.

Scientific studies have shown that in the natural product produced according to EP 1 153 549 B1 saturation or decay kinetics occur with regard to the immune stimulation. This loss of activity within a higher concentration range may be compensated in that live probiotic microorganisms are added to the natural product. However, it has been found that the probiotic microorganisms become inactive within a short period of time, i.e. within a few hours, which is attributed to the acidic environment of the natural product.

The object of the present invention is therefore to provide a method for the production of a fermented natural product, which is highly effective over a long period of time, even at high concentrations, and may be stored for a long period of time without any problems, without weakening the enzyme controlling activity of the natural product.

Another object of the present invention is to provide a system, by means of which the method according to the invention may be carried out efficiently and economically.

### DESCRIPTION OF THE INVENTION

The task of the present invention is solved by means of a method for the production of a fermented product according to claim 1.

The present invention relates to a method for the production of a fermented natural product, comprising the steps of:
a) fermenting raw materials for the natural product in the presence of microorganisms in a fermenter for the production of a first ferment extract;
b) removing at least one part of the first ferment extract and transferring said part to at least one additional fermenter;
c) fermenting said part of the ferment extract in the presence of microorganisms for forming at least one partial ferment extract;
d) mixing said at least one partial ferment extract with the first ferment extract, and
e) adding a propagate of microorganisms to the combined ferment extracts.

The present invention also relates to a system, in particular for the production of a fermented natural product according to claim 16.

The present invention relates to a system, in particular for the production of a fermented natural product, having at least two fermenters and one propagator.

The sub-claims relate to preferred embodiments of the method according to the invention and of the system according to the invention.

The method according to the invention has been successful in providing a concentrated, fermented natural product significantly activating the enzyme of the immune defense of the body. This characteristic remains intact even after rendering the same non-perishable by means of short-term heat treatment. The filled fermented natural product may be stored for a longer period of time without any problems, without the natural product losing any of its activity.

### BRIEF DESCRIPTION OF THE DRAWINGS

The present invention is explained in further detail with reference to the drawings. They show:

FIG. 1 a process diagram explaining one embodiment of the method according to the invention according to variation A;

FIG. 2 a process diagram for one embodiment of process variation B of the method according to the invention;

FIG. 3 a diagram describing the immune-modulating activity of a natural product according to prior art, and

FIG. 4 a diagram showing the immune-modulating activity of the products produced according to the invention;

FIG. 5 a diagram showing the immune-modulating activity of the products produced according to the invention having a natural product according to prior art.

### DETAILED DESCRIPTION OF THE INVENTION

The method according to the invention provides a fermented natural product, which retains its immune-modulating activities over a long period of time due to the enrichment of the same with microorganisms in the combined ferment extracts. Even after heat deactivation, i.e. in the heat deactivated state, the added microorganisms bring about additive effects of the activation with the also heat-deactivated microorganisms from the fermentation. These effects have been experimentally verified as follows.

For carrying out the tests for detecting immune-modulating activities, the ACC/whole blood test system was used. These test systems simulate an inflammatory reaction by means of activation of neutrophil granulocytes in whole blood.

The test is based on the fact that the formation of activated neutrophil granulocytes may be verified in fresh human blood in the presence of immune activators. The granulocytes contained in blood recognize the immune activators and are transferred into an activated state. In the activated state the neutrophil granulocytes produce active oxygen species, wherein hydrogen peroxide is converted with chloride into sub-chloride acid (HOCl) by the enzyme myeloperoxidasis (MPO). The sub-chloride acid is then reacted into ethene and other products with 1-aminocyclopropane-1-carboxyl acid (ACC) as the indicator molecule. The ethane formed is a measure for the activated inflammatory reaction and is gaschromatically verified.

In the biochemical model the whole blood is incubated for 30 minutes with the substances to be tested using the indicator molecule ACC in volume-calibrated, hermetically sealed reaction vessels. Subsequently, the ethene released by means of ACC is gaschromatically quantified.

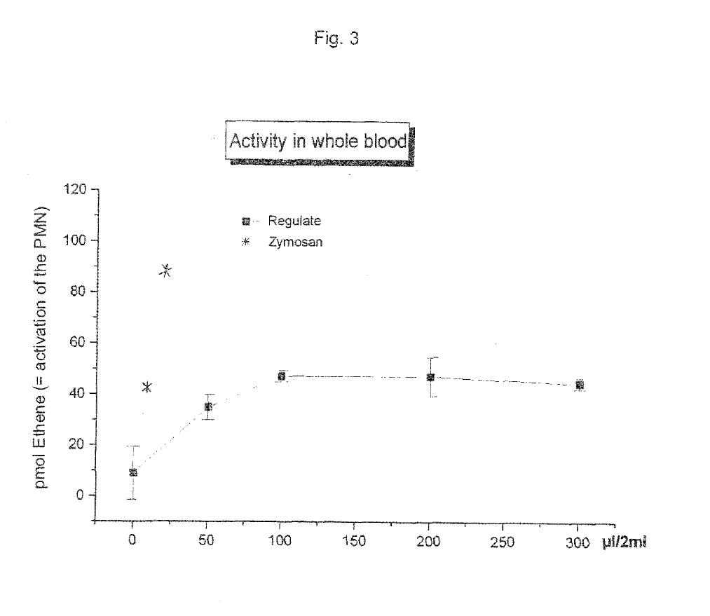

FIG. 3 shows a diagram for the activation of neutrophil granulocytes in whole blood. The immune-modulating activity of the regulate, a natural product produced in accordance with EP 1 153 549 B1, was measured. As a reference, a sample was additionally tested with Zymosan (a cell wall preparation made from yeast), which is an experimental activator of the innate immune response (innate immunity). The concentration of heat-activated dead microorganisms is within a range of 0 to 300 μl/2 ml of whole blood. This corresponds to an amount of 5×10¹⁰ to 2.5×10¹¹ of cells. The Zymosan was tested as Zymosan 50 with 200 μg/2 ml of whole blood.

As obvious from FIG. 3, an activation of the immune reaction occurred in the regulate, which jumps to 100 μl/2 ml of whole blood, subsequent to which a saturation or a decay, respectively, of the immune reaction occurs at higher concentrations. In the case of Zymosan a jump in activation of the immune response expectantly occurs already at the lowest amounts. The binding of ethene is measured in pmol/60 minutes.

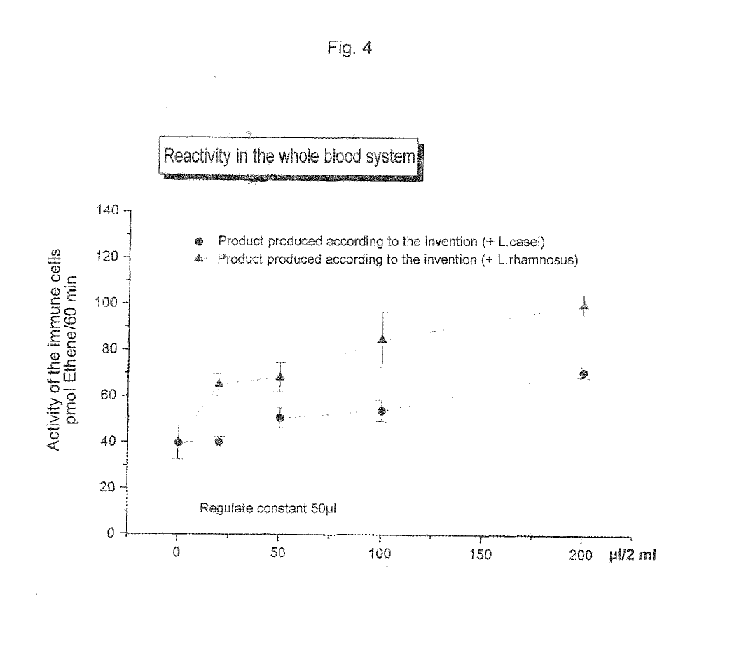

The diagram of FIG. 4 shows the immune-stimulating reactivity of two products produced according to the invention in the whole blood system. The point-dotted curve shows a product produced according to the invention, to which a propagate of Lactobacillus casei has been added to the combined ferment extracts after fermentation. The triangular-dotted curve shows a further product produced according to the invention, to which a propagate of Lactobacillus rhamnosus has been added after fermentation. In the increase of the formation of ethene it is clear that with an increasing concentration additive effects of the immune activation are brought about. The saturation or decay kinetics, respectively, determined in FIG. 3 with the product according to prior art, is completely removed. In particular at 100 to 200 μl/2 ml of whole blood a significant increase of immune response is determined. Therefore, a continuous increase in activity is evident, which is not shown in both microorganisms alone.

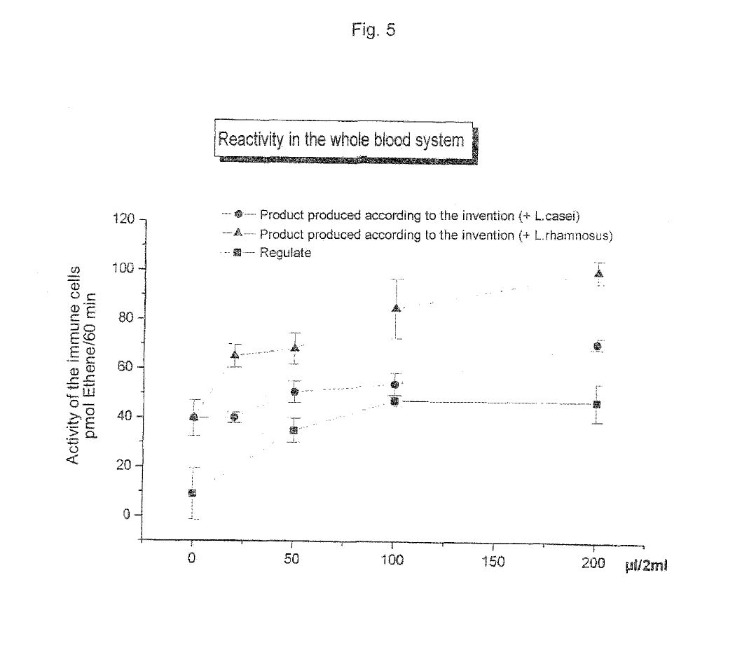

FIG. 5 shows the reactivities in the whole blood system in a direct comparison. As is obvious, the natural products produced according to the invention achieve an activity increased over a centration range of 0 to 200 μl/2 ml, while the immune-modulating activity of the product according to prior art stagnates, and even decreases above 100 μl/2 ml. It has been determined that the addition of Lactobacillus rhamnosus to the propagate has a stronger influence on the immune-stimulating effect, than Lactobacillus casei.

After the addition of the propagate from the microorganisms to the combined ferment extracts, the ferment extracts containing microorganisms may be heat-treated at a temperature within a range of 65° C. to 75° C., in order to deactivate the microorganisms contained therein. The natural product produced according to the method according to the invention therefore does not contain any microorganisms which may proliferate. In a particular embodiment of the present invention the heat treatment is carried out at a temperature within a range of 68° C. to 72° C. This heat treatment is a short-term heating preferably carried out within a time period of 60 to 360 seconds, preferably 120 to 300 seconds.

After adding the propagate, and, if necessary, after the short-term heating the final product is guided into a filling device, where it is then filled into suitable containers, such as bottles. The bottles are then hermetically sealed in a commonly known manner.

The combined ferment extracts having the microorganisms contained therein, have a pH of 3.2 to 4.

In a preferred embodiment of the method according to the invention the microorganisms are added in steps a) and c) at an amount of 10⁶ to 10¹² cells/ml, preferably 10⁸ to 10¹⁰ cells/ml.

It has been shown to be advantageous for the method according to the invention, if the propagate from microorganisms is added to the combined ferment extracts in step e) at an amount, which approximately leads to the doubling of the number of microorganisms.

The propagate from microorganisms is cultivated in a propagator. Usually, the microorganisms are initially cultivated in a starter culture in the propagator, and then cultivated to a concentration of 10¹² to 10¹⁶ cfu/ml, preferably 10¹³ to 10¹⁵ cfu/ml. Up to two thirds of the propagate is guided into the filling tank 8, where the combined ferment extracts are located.

By adding substrate, nutrients are provided for the remaining propagate in order to achieve a renewed propagation of the microorganism culture to the target concentration. As a substrate, the raw materials for the natural product may be added to the microorganisms in the propagator in a crushed and clarified form. As an alternative, a culture medium may be added to the microorganisms in the propagator. In this case an additional container is provided, containing the culture medium, which is then guided into the propagator in a controlled manner. Oxygen is also fed into the propagator, in order to create aerobic conditions within the propagator. This may occur, for example, by means of introducing air into a ventilator device.

According to the invention, fruit, vegetables, legumes, mushrooms, nuts, wheat, rice, herbs, roots, leaves, blossoms, are used as raw materials, either individually or in combination. The mixture itself is considered non-critical. Practically any variation of fruit, vegetables, legumes, mushrooms, nuts, wheat, rice, herbs, roots, leaves, blossoms, is possible. One preferred mixture for the method according to the invention is a mixture of fruit, vegetables, legumes, nuts, and herbs.

The raw materials may be used, for example, in an amount of 300 kg, based on 1000 liters of preparation. Before fermentation they are crushed in a commonly known manner, such as by means of cutting or shredding. The raw materials used preferably come from biological crops.

Depending on the type of natural product to be produced, the same or different microorganisms may be used in the fermenting steps a) and c), as well as with the propagate addition. In a preferred embodiment microorganisms are used, which are selected from bacteria, fungi, yeasts, and/or mixtures thereof. The use of bacteria is particularly preferred. In principle, any bacterium may be used for the fermentation, or the propagate addition, respectively, as long as it corresponds with the food regulations. In a preferred embodiment of the method according to the invention, bacteria selected from the species of

Lactobacillus are used, Examples of this are Lactobacillus casei and Lactobacillus rhamnosus. In a further embodiment the Lacto-bacteria may be used in a mixture together with other bacteria, i.e. such bacteria that are not Lacto-bacteria. Preferably, probiotic bacteria are used.

Usually a culture medium, such as a lactose solution, is provided as the substrate for the microorganisms.

The method according to the invention comprises at least two process variations A and B, described in further detail below.

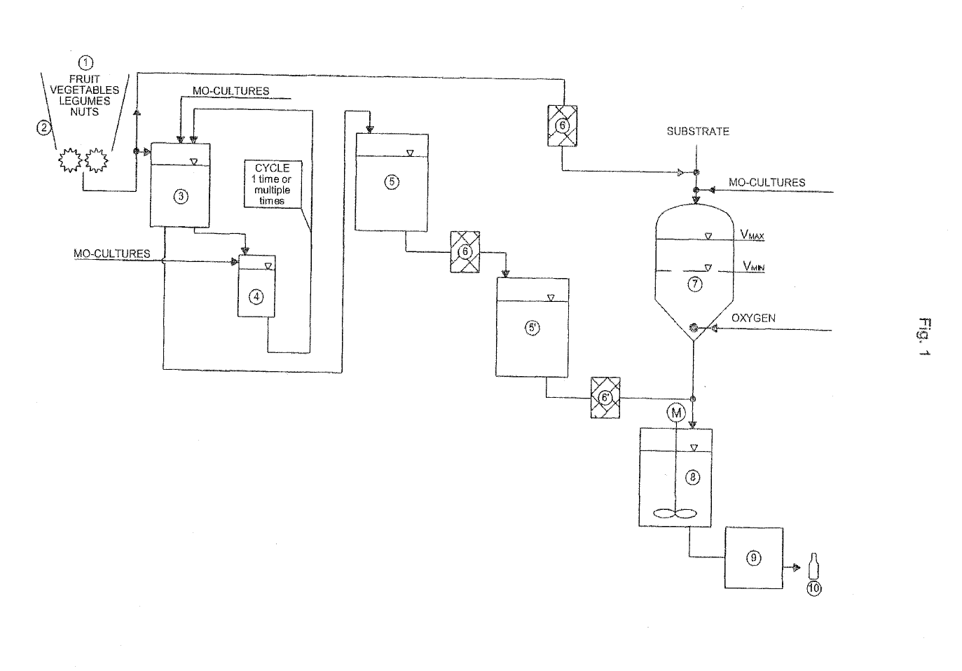

The process variation A is explained in further detail in FIG. 1. FIG. 1 shows a process diagram for an embodiment of variation A of the method according to the invention. In this process variation all partial ferment extracts come from the first ferment extract, which are then combined with the first ferment extract.

The crushed raw materials, such as fruit, vegetables, legumes and nuts, are located in a container for raw materials 2, from which the raw materials are guided into a main fermenter 3. Subsequently, the microorganism cultures are then added to the main fermenter. Fermentation is generally carried out at a temperature within a range of 20° C. to 35° C., preferably 25° C. to 32° C. The temperature depends on which microorganism or which microorganism mixture is used. Generally, the temperature is 28° C., wherein normally fermentation takes place within a time period of 10 to 20 days, depending on the preparation and the microorganisms used.

From this preparation at least one part, such as up to one third, is taken and transferred into at least one additional fermenter, the auxiliary fermenter 4. Fermentation is again effected in this auxiliary fermenter while adding microorganisms at a temperature of 20° C. to 35° C. Again, the temperature also depends on which microorganism is used. Normally, the temperature is 28° C. After completed partial fermentation, said preparation is again transferred from the auxiliary fermenter 4 into the main fermenter 3. This cycle may be repeated several times.

Variation A is therefore based on the principle that all partial ferment extracts come from the first ferment extract, and the partial fermentations are combined with the first ferment extract.

After all partial fermentations having the first ferment extract have been transferred into a collection tank 5, the same are filtered, if necessary, and received in an additional collection tank 5′. Further clarification by means of filtration is carried out in the filter 6′ in a subsequent filtration unit. The clarified filtrate is transferred into a filling tank 8, into which a propagate of fresh microorganisms is introduced from a propagator 7. After mixing, for example, by means of stirring, in the filling tank 8, the ferment extracts comprising the microorganisms are filled in a filling device 9 into containers 10, e.g. bottles, in a commonly known manner.

If necessary, the fermentation extracts having the microorganisms are treated in a heater (not shown) at a temperature of 65° C. to 75° C., preferably within a time period of 60 to 360 seconds, preferably 120 to 300 seconds. The short-term heating serves to deactivate the microorganisms in the final product.

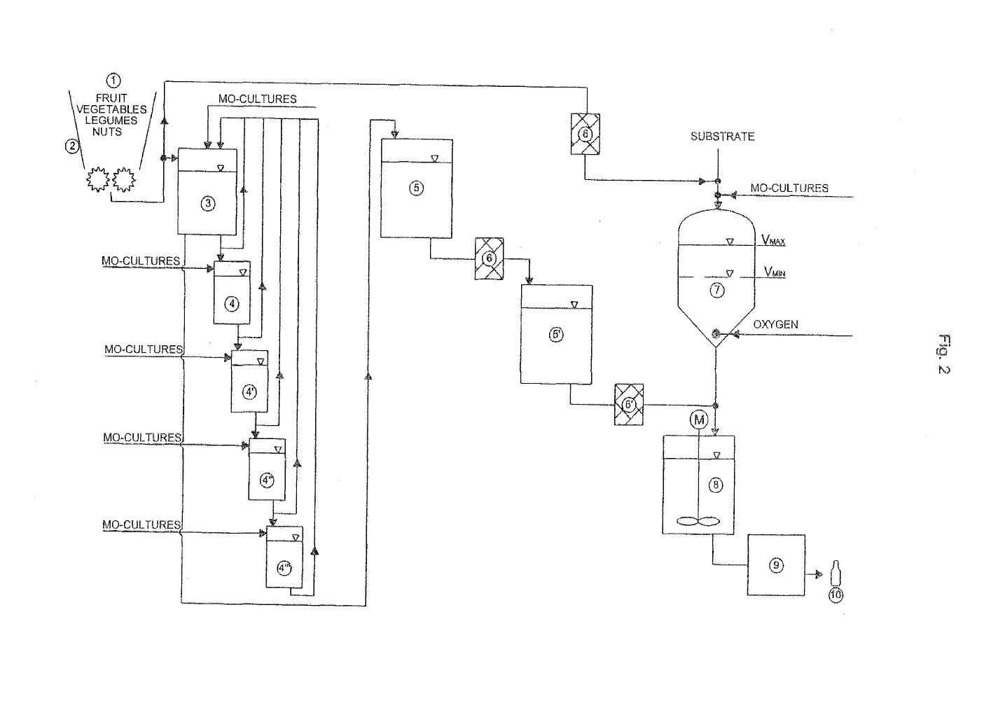

The second process variation B is illustrated in FIG. 2. In this process variation, as with the process variation A, the first partial ferment extract comes from the first ferment extract, wherein additional partial fermentations occur in parallel connected auxiliary fermenters, and the individual partial fermentations are combined with the still remaining first ferment extract. The crushed raw materials in the container 2 are transferred into the main fermenter 3, in that a first ferment extract is produced by means of the addition of microorganisms. With regard to the process parameters, reference is made to the explanations for process variation A. A part of the first ferment extract, such as up to one third, is then taken from the main fermenter 3 and transferred to a first auxiliary fermenter 4′, wherein the fermentation is carried out in the presence of microorganisms for the formation of at least one partial ferment extract. After fermentation, a part is again taken from the auxiliary fermenter 4′ and transferred to the auxiliary fermenter 4″, where again a partial fermentation is carried out in the presence of additional microorganisms. Said partial fermentations may be repeated in any number of additional auxiliary fermenters, such as in an auxiliary fermenter 4′″.

The individual partial fermentations from the auxiliary fermenters 4′, 4″, and 4′″ are again transferred to the main fermenter 3. The combined ferment extracts are then transferred from the same to a collection tank 5, subsequent to which the same is then filtered and a propagate of microorganisms is added, as described above for the process variation A. Reference is also made to the description of process variation A with regard to the further course of the method.

In this process variation the auxiliary fermenters are connected to each other, i.e. are series-connected. This variation B differs from variation A in that larger molecular structures may remain intact in addition to smaller molecular structures.

With both variations any number of partial fermentations may be carried out. It is assumed that a natural product produced from multiple partial fermentations has a particularly unusual, multi-functional breadth of effectiveness, in particular in immune modulating.

It is furthermore advantageous to add additives to the combined ferment extracts containing the microorganisms before filling. As an alternative, said additives may also be added before the collection container 5′.

These additives include, for example, secondary plant substances, such as luteins, vitamins, minerals, fatty acids, polysaccharides, polyglucanes, and/or extracts from fungi. The composition of these additives is carried out individually, and depends on the desired spectrum of effectiveness. For example, the circle of consumers plays a role in the selection of the individual components. While women have an increased need for folic acid, iron, magnesium, zinc, and biotin, the substitution of vitamin K, B5, B9, B2 is suitable for men. Athletes have an increased demand for sodium, calcium, magnesium, zinc, vitamin D and C.

The product produced according to the invention has the advantage that it strengthens the immune defense at a low dosage, and comprises a particularly high antioxidative potential. The product is durable for a time period of at least three years in a hermetically sealed state.

The natural product produced according to the invention is a bio-product and may be used for oral and external administration. For oral administration, it is generally administered orally as a food products or edibles, respectively, or as a pharmaceutical product. It may be presented in the form of a liquid. It is also possible to form a lyophilisate from it, which may be dissolved, if necessary, in water or another liquid. It may also be advantageous to administer the natural product according to the invention in the form of tablets, pastilles, granulates, vials, drops, or sprays.

The natural product produced according to the invention may also be applied topically on the skin. In this regard it has been shown to be favorable, if the natural product, which may be present, for example, in the form of a liquid, is added to dressings or compresses, and then placed onto the affected skin locations.

The natural product produced according to the invention may also be used as a cosmetic product, such as incorporated in creams, ointments, and foams, thus being directly applied to the skin. The cosmetic products have moisturizing properties, and act as anti-aging agents.

The system according to the invention is suitable for carrying out the method according to the invention. This system can be used to carry out both process variations.

The system 1 according to the invention has at least two fermenters 3; 4, and a propagator 7 for cultivating microorganisms. The individual devices of the system according to the invention are described in the process diagrams of FIGS. 1 and 2.

In a preferred embodiment of the system 1 according to the invention the at least two fermenters consist of one main fermenter 3 and at least one auxiliary fermenter 4. A first ferment extract is produced in the main fermenter 3. At least one additional partial ferment extract is produced in at least one auxiliary fermenter 4. For process variation B the auxiliary fermenters 4; 4′; 4″; 4′″ are series-connected and thus connected to each other. If necessary, the system also comprises sedimentation devices after the main fermenter and the auxiliary fermenters, in order to clarify the ferment extracts.

The system according to the invention further comprises a collection tank 5, into which the combined ferment extracts are guided. A filter 6 cleaning the ferment extracts, or frees the same from insoluble components, is connected downstream of the collection tank 5. The clarified ferment extracts are then again collected in a collection tank 5′, and then in turn guided into a filter device 6′ for further clarification.

The system 1 according to the invention comprises a propagator 7, in which fresh microorganisms are cultivated. This propagator 7 is connected to supply lines guiding the substrate and microorganism culture into the propagator,

In one embodiment of the system according to the invention a partial stream is branched off after the container for raw materials 2 as the substrate for the propagate in the propagator 7.

The system according to the invention further comprises preferably a filling tank 8 having a stirring device, by means of which the fresh microorganisms and the clarified, combined ferment extracts discharged from the propagator are homogenously mixed.

The system 1 may comprise a heater (not shown), in which the ferment extracts having the microorganisms from the propagator 7 are heated to a temperature of 65° C. to 75° C., preferably 68° C. to 72° C.

A filling device 9 serves to fill the optionally heated natural product into suitable containers, such as bottles 10.

The bottles are then hermetically sealed immediately after filling.

1-20. (canceled)

21. Method for the production of a fermented natural product, comprising the steps of:
a) fermenting raw materials for the natural product, selected from fruit, vegetables, legumes, mushrooms, nuts, wheat, rice, herbs, roots, leaves, blossoms, either individually or in combination, in the presence of microorganisms in a main fermenter (3) for the production of a first ferment extract;
b) removing at least one part of the first ferment extract and transferring said part to at least one additional auxiliary fermenter (4; 4′; 4″; 4′″), wherein the part taken forms up to one third of the first ferment extract;
c) fermenting said part of the ferment extract in the presence of microorganisms for forming at least one partial ferment extract;
d) transferring said at least one partial ferment extract to the main fermenter (3), and mixing with the remaining first ferment extract, and
e) adding a propagate of microorganisms to the combined ferment extracts, wherein the microorganisms in steps a) and c) are added at an amount of 10⁶ to 10¹² cells/ml, preferably 10⁸ to 10¹⁰ cells/ml.

22. Method according to claim 21, characterized in that the propagate of microorganisms is added to the combined ferment extracts at an amount approximately leading to the doubling of the number of microorganisms.

23. Method according to claim 21, characterized in that the product is filled into suitable containers.

24. Method according to claim 21, characterized in that all partial ferment extracts come from the first ferment extract and the partial fermentations are combined with the first ferment extract.

25. Method according to claim 21, characterized in that the first partial ferment extract comes from the first ferment extract, and additional partial fermentations are carried out in parallel connected fermenters, and the individual partial fermentations are combined with the first ferment extract.

26. Method according to claim 21, characterized in that the propagate is cultivated from microorganisms in a propagator.

27. Method according to claim 26, characterized in that the raw materials for the natural product are added as a substrate to the microorganisms in the propagator.

28. Method according to claim 26, characterized in that a culture medium is added as a substrate to the microorganisms in the propagator.

29. Method according to claim 21, characterized in that the same or different microorganisms are used in the fermentation steps a) and c), as well as with the propagation addition.

30. Method according to claim 21, characterized in that bacteria are used as the microorganisms, which are selected from the species of Lactobacillus.

31. Method according to claim 20, characterized in that the Lacto-bacteria are used in a mixture together with other bacteria.

32. Method according to claim 21, characterized in that the combined ferment extracts are heat-treated with the propagate at a temperature within a range of 65° C. to 75° C., preferably 68° C. to 72° C.

33. Method according to claim 32, characterized in that the combined ferment extracts are heat-treated for a short period of time of 60 to 360 seconds, preferably 120 to 300 seconds.

34. System (1), in particular for the production of a fermented natural product, comprising
at least two fermenters (3; 4), and
one propagator (7)
a filling device (9).

35. Method according to claim 34, wherein the at least two fermenters consist of one main fermenter (3) and more than one auxiliary fermenter (4) characterized in that the auxiliary fermenters (4; 4′; 4″; 4′″) are series-connected.

36. Method according to claim 34, characterized in that supply lines are connected to the propagator (7), which guide microorganism cultures into the propagator.

37. Method according to claim 34, characterized in that it further comprises a heater, in which the combined ferment extracts having the microorganisms from the propagator (7) are heated to a temperature of 65° C. to 75° C., preferably 68° C. to 72° C.
